(19) United States
(12) Patent Application Publication
(10) Pub. No.: US 2024/0325262 A1
(43) Pub. Date: Oct. 3, 2024

(54) FLUORAPATITE GLASS-CERAMICS

(71) Applicant: CORNING INCORPORATED, Corning, NY (US)

(72) Inventor: Qiang Fu, Painted Post, NY (US)

(21) Appl. No.: 18/613,825

(22) Filed: Mar. 22, 2024

Related U.S. Application Data
(60) Provisional application No. 63/455,475, filed on Mar. 29, 2023.

(51) Int. Cl. A61K 8/21; A61K 8/19; A61K 8/24; A61K 8/25; A61K 8/28; A61Q 11/00; C03B 32/02; C03C 4/00; C03C 10/16

(52) U.S. Cl. CPC A61K 8/21; A61K 8/19; A61K 8/24; A61K 8/25; A61K 8/28; A61Q 11/00; C03B 32/02; C03C 4/0021; C03C 10/16; C03C 2204/00

(57) ABSTRACT

A glass composition and method of making, the glass composition comprising 15-45 wt. % SiO₂, 10-30 wt. % P₂O₅, 30-60 wt. % CaO, 0.5-15 wt. % ZrO₂, and 0.1-10 wt. % F. In some aspects, the glass composition comprises a glass-ceramic comprising a fluorapatite crystal phase. A matrix comprising the glass composition, in which the matrix is a dentifrice composition in some aspects. A method for applying the matrix to a tooth, in which the method remineralizes enamel of a tooth, treats caries of a tooth, treats dentin hypersensitivity of the tooth. A method of making the matrix comprising combining a carrier with the glass composition.

### CROSS-REFERENCE TO RELATED APPLICATIONS

This application claims the benefit of priority of U.S. Provisional Application Ser. No. 63/455,475, filed on Mar. 29, 2023, the content of which is relied upon and incorporated herein by reference in its entirety.

### FIELD

This disclosure generally relates to glass compositions, and more particularly to bioactive glass compositions for consumer, dental, and cosmetic applications.

### BACKGROUND

Bioactive glasses are a group of glass and/or glass ceramic materials that have shown biocompatibility or bioactivity, which has allowed them to be incorporated into human or animal physiology. Generally speaking, bioactive glasses may be able to bond with hard and soft tissues, thereby fostering growth of bone and cartilage cells. Moreover, bioactive glasses may also enable release of ions which activate expression of osteogenic genes and stimulate angiogenesis, as well as promote vascularization, wound healing, and cardiac, lung, nerve, gastrointestinal, urinary tract, and laryngeal tissue repair.

Some bioactive glasses, such as 45S5 Bioglass® sold by Haleon under the Sensodyne® brand featuring NovaMin® technology, are being investigated for their potential ability to mineralize teeth; however, the low chemical durability of these traditional bioactive glasses is problematic for compositions requiring prolonged shelf times in aqueous environments. For example, 45S5 Bioglass® requires development of a non-aqueous environment for glass particulates to be used in toothpaste applications. In addition, the speed at which 45S5 Bioglass® may mineralize teeth also is not ideal.

Thus, there is a need for improved bioactive glass compositions.

### SUMMARY

In some aspects, disclosed are glass compositions comprising:
15-45 wt. % SiO₂;
10-30 wt. % P₂O₅;
30-60 wt. % CaO;
0.5-15 wt. % ZrO₂;
0.1-10 wt. % F⁻;
0-25 wt. % MgO;
0-5 wt. % B₂O₃;
0-5 wt. % Al₂O₃;
0-10 wt. % Li₂O;
0-10 wt. % Na₂O;
0-10 wt. % K₂O;
0-10 wt. % SrO;
0-10 wt. % ZnO; and
Li₂O+Na₂O+K₂O of less than or equal to 10 wt. %;
based on total weight of the glass composition.

In some aspects, the glass composition is a glass-ceramic comprising a fluorapatite crystal phase. In some aspects, the glass composition further comprises at least one minor crystalline phase of calcium silicate, calcium phosphate, calcium fluoride, or any combination thereof.

In some aspects, disclosed is a matrix comprising the glass composition. In some aspects, the matrix is a dentifrice composition, such as toothpaste.

In some aspects, disclosed is a method comprising applying to a tooth a dentifrice composition comprising the glass composition. In some aspects, the method remineralizes enamel of a tooth, treats caries of a tooth, treats dentin hypersensitivity of the tooth, or any combination thereof.

In some aspects, disclosed is a method for making the glass composition, comprising:
combining each component of the glass composition to form a mixture,
heating the mixture to a temperature of 1500° C. or less to form a melted mixture, and
cooling the melted mixture to form a cooled mixture.

Additional features and advantages of the disclosure will be set forth in the detailed description which follows, and in part will be readily apparent to those skilled in the art from that description or recognized by practicing the aspects as described herein, including the detailed description which follows, the claims, as well as the appended drawings.

It is to be understood that both the foregoing general description and the following detailed description are merely exemplary and are intended to provide an overview or framework for understanding the nature and character of the disclosure and claims. The accompanying drawings are included to provide a further understanding of the disclosure and are incorporated into and constitute a part of this specification. The drawings illustrate various aspects of the disclosure and together with the description serve to explain the principles and operations of the various aspects.

### BRIEF DESCRIPTION OF THE DRAWINGS

The following detailed description can be further understood when read in conjunction with the following drawings. Whenever possible, the same reference numerals will be used throughout the drawings to refer to the same or like parts. It is to be understood that the figures are not drawn to scale and the size of each depicted component or the relative size of one component to another is not intended to be limiting.

FIG. 1 is a powder X-ray diffraction (XRD) spectrum illustrating formation of a fluorapatite glass-ceramic upon cooling a melted glass mixture without a subsequent heat treatment step.

### DETAILED DESCRIPTION

In the following description, whenever a group is described as comprising at least one of a group of elements and combinations thereof, it is understood that the group may comprise, consist essentially of, or consist of any number of those elements recited, either individually or in combination with each other. Similarly, whenever a group is described as consisting of at least one of a group of elements or combinations thereof, it is understood that the group may consist of any number of those elements recited, either individually or in combination with each other.

Where a range of numerical values is recited herein, comprising upper and lower values, unless otherwise stated in specific circumstances, the range is intended to include the endpoints thereof, and all integers and fractions within the range. Further, when an amount, concentration, or other value or parameter is given as a range, one or more ranges, or a list of upper values and lower values, this is to be understood as specifically disclosing all ranges formed from any pair of any upper range limit or value and any lower range limit or value, regardless of whether such pairs are separately disclosed.

If the term “about” is used in describing a value or an end-point of a range, the disclosure should be understood to include the specific value or end-point referred to. As used herein, the term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. It is noted that the terms “substantially” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue. Thus, for example, a glass that is “free” or “essentially free” or “substantially free” of Al₂O₃ (or any other component) is one in which Al₂O₃ (or any other component) is not actively added or batched into the glass, but may be present in very small amounts as a contaminant (e.g., 500, 400, 300, 200, or 100 ppm or less).

As used herein, a glass, glass-ceramic, or crystalline phase that is “bioactive” means that the glass, glass-ceramic, or crystalline phase is biologically compatible with bone, teeth, and/or tissue. By way of illustration, in some aspects, “bioactive” in this context generally refers to the capability of binding with a desired biological material (e.g., bones, teeth, and/or tissue). Generally, there is also an absence of toxicity or other significant negative effects in a biological environment (e.g., bones, teeth, and/or tissue).

Herein, glass compositions are expressed in terms of wt. % amounts of particular components included therein on an oxide bases unless otherwise indicated. Any component having more than one oxidation state may be present in a glass composition in any oxidation state. However, concentrations of such component are expressed in terms of the oxide in which such component is at its lowest oxidation state unless otherwise indicated.

Oral diseases pose a major health burden worldwide, causing pain, discomfort, disfigurement, and even death. The dissolution of apatite crystals and the net loss of calcium, phosphate, and other ions from the tooth (i.e., demineralization) leads to dental caries formation. Caries can be managed non-invasively through a remineralization process, in which calcium and phosphate ions are supplied from an external source to the tooth to promote crystal deposition into voids in demineralized enamel. Calcium phosphate phases in both crystalline form (e.g., brushite, β-tricalcium phosphate, octocalcium phosphate, hydroxyapatite, fluorapatite and enamel apatite) and amorphous form have been used in remineralization processes. Use of amorphous calcium phosphate (e.g., bioactive glass) in remineralization processes has shown promising results.

Fluorapatite in particular shows great promise as an ingredient in consumer compositions, such as dentifrice compositions, since fluorapatite is a naturally occurring mineral of biological and agricultural importance, as well as an inorganic component in human and animal hard tissues, such as bones and teeth.

There is a strong desire to develop new glass compositions, and methods of making, that promote the remineralization process to, for example, prevent or repair/treat tooth caries, to prevent or repair/treat dentin sensitivity.

Bioactive glasses are a group of glass and/or glass ceramic materials that have shown biocompatibility or bioactivity, which has allowed them to be incorporated into human or animal physiology. In some aspects, disclosed is a glass composition, e.g., a bioactive glass composition, comprising SiO₂, CaO, and P₂O₅. In some aspects, the glass composition further comprises ZrO₂ and/or F⁻. In some aspects, the glass composition is a glass-ceramic. In some aspects, when melting precursor oxides at a temperature of 1500° C. or less (e.g., 1400° C. or less), the composition spontaneously forms a crystalline phase upon cooling (e.g., one or more of fluorapatite, calcium silicate, calcium phosphate, calcium fluoride, or any combination thereof), without the need to subject the cooled composition to a subsequent heat treatment step. However, in some aspects, it may be desirable to subject the cooled composition to a subsequent heat treatment step, for example, to tune the amounts and types of crystalline phase present.

In some aspects, the glass composition comprises a combination of SiO₂, CaO, and P₂O₅. In some aspects, the glass composition further comprises ZrO₂. In some aspects, the glass composition further comprises F. In some aspects, the glass composition further also comprises MgO, SrO, ZnO, or any combination thereof. In some aspects, the glass composition further comprises Li₂O, Na₂O, K₂O, or any combination thereof. In some aspects, the amount of Li₂O+Na₂O+K₂O is less than or equal to 10 wt. %. In some aspects, the glass composition further comprises Al₂O₃, B₂O₃, or a combination thereof.

In some aspects, the glass composition comprises SiO₂, CaO, P₂O₅, ZrO₂, and F.

In some aspects, the glass composition comprises SiO₂ in an amount of 15-45 wt. %. In some aspects, the glass composition comprises CaO in an amount of 30-60 wt. %. In some aspects, the glass composition comprises P₂O₅ in an amount of 10-30 wt. %. In some aspects, the glass composition comprises ZrO₂ in an amount of 0.5-15 wt. %. In some aspects, the glass composition comprises F in an amount of 0.1-10 wt. %.

In some aspects, the glass composition comprises:
15-45 wt. % SiO₂;
10-30 wt. % P₂O₅;
30-60 wt. % CaO;
0.5-15 wt. % ZrO₂;
0.1-10 wt. % F⁻;
0-25 wt. % MgO;
0-5 wt. % B₂O₃;
0-5 wt. % Al₂O₃;
0-10 wt. % Li₂O;
0-10 wt. % Na₂O;
0-10 wt. % K₂O;
0-10 wt. % SrO;
0-10 wt. % ZnO; and
Li₂O+Na₂O+K₂O of less than or equal to 10 wt. %;
based on total weight of the glass composition.

In some aspects, the glass compositions comprise:
25-45 wt. % SiO₂;
10-25 wt. % P₂O₅;
37-50 wt. % CaO;
0.5-8 wt. % ZrO₂;
0.1-5 wt. % F;
>0-10 wt. % MgO;
based on total weight of the glass composition.

In some aspects, the glass compositions comprise:
at least one of:
   >0-25 wt. % MgO;
   >0-5 wt. % B₂O₃;
   >0-5 wt. % Al₂O₃;
   >0-10 wt. % Li₂O;
   >0-10 wt. % Na₂O;
   >0-10 wt. % K₂O;
   >0-10 wt. % SrO; or
   >0-10 wt. % ZnO;
provided that Li₂O+Na₂O+K₂O is less than or equal to 10 wt. %;
based on total weight of the glass composition.

Silicon dioxide (SiO₂) may serve as the primary glass-forming oxide component of the glass compositions disclosed herein. SiO₂ may be included to provide high temperature stability and chemical durability. However, if too much SiO₂ is included, such as a glass containing pure SiO₂, the melting temperature is too high to be readily workable (e.g., greater than 200 poise temperature). In addition, glass compositions including too much SiO₂ may suffer from decreased bioactivity.

In some aspects, the glass compositions comprise SiO₂ in an amount of at least 15 wt. %. In some aspects, the glass compositions comprise SiO₂ in an amount of 45 wt. % or less. In some aspects, the glass compositions comprise SiO₂ in an amount (wt. %) of 15-45, 15-40, 15-35, 15-30, 15-25, 15-20, 20-45, 20-40, 20-35, 20-30, 20-25, 25-45, 25-40, 25-35, 25-30, 30-45, 30-40, 30-35, 35-45, 35-40, or 40-45.

Phosphorus pentoxide (P₂O₅) may serve as a network former in glass compositions. Furthermore, the liberation of phosphate ions to the surface of bioactive glasses contributes to the formation of apatite. Apatite is an inorganic mineral in bone and teeth. The inclusion of phosphate ions in the bioactive glass increases apatite formation rate and the binding capacity of the hard tissues (e.g., bone, tooth, etc.). In addition, P₂O₅ can increase the viscosity of the glass, which in turn expands the range of operating temperatures, and is therefore an advantage to the manufacture and formation of the glass.

In some aspects, the glass compositions comprise P₂O₅ in an amount of at least 10 wt. %. In some aspects, the glass compositions comprise P₂O₅ in an amount of 30 wt. % or less. In some aspects, the glass compositions comprise P₂O₅ in an amount (wt. %) of 10-30, 10-25, 10-20, 10-15, 15-30, 15-25, 15-20, 20-30, 20-25, or 25-30.

Zirconium dioxide (ZrO₂) may also be present and serves to function as a network former or intermediate in precursor glasses, as well as a key oxide for improving glass thermal stability by significantly reducing glass devitrification during forming and lowering liquidus temperature. In aspects, ZrO₂ may play a similar role as alumina (Al₂O₃) in the composition.

In some aspects, the glass compositions comprise ZrO₂ in an amount of at least 0.5 wt. %. In some aspects, the glass compositions comprise ZrO₂ in an amount of 15 wt. % or less. In some aspects, the glass compositions comprise ZrO₂ in an amount (wt. %) of 0.5-15, 0.5-14, 0.5-12, 0.5-10, 0.5-8, 0.5-6, 0.5-4, 0.5-2, 0.5-1, 1-15, 1-14, 1-12, 1-10, 1-8, 1-6, 1-4, 1-2, 2-15, 2-14, 2-12, 2-10, 2-8, 2-6, 2-4, 4-15, 4-14, 4-12, 4-10, 4-8, 4-6, 6-15, 6-14, 6-12, 6-10, 6-8, 8-15, 8-14, 8-12, 8-10, 10-15, 10-14, 10-12, 12-15, 12-14, or 14-15.

In some aspects, the glass compositions comprise fluoride (F⁻), which facilitates remineralization of enamel. Such remineralization can form bioactive crystalline phases, such as hydroxyapatite, fluorapatite, carbonated apatite, or any combination thereof. In some aspects, at least some of such bioactive crystalline phases are resistant to acid corrosion, such as fluorapatite. F⁻ can combine with CaO and P₂O₅ to form fluorapatite to improve the bioactivity of the glass compositions. Fluorapatite is an inorganic mineral in dental enamel. The ability to form fluorapatite can help regeneration of the enamel that has become demineralized, for example, due to tooth caries (e.g., cavities). In some aspects, formation of apatite ensure the high bioactivity of the compositions disclosed herein. Moreover, without wishing to be bound by theory, it is believed that F⁻ may play a role in facilitating crystallization of the glass composition upon cooling, for example, into one or more crystalline phases such as fluorapatite, calcium silicate, calcium phosphate, calcium fluoride, or any combination thereof.

In some aspects, the F⁻ in the glass compositions is derived from calcium fluoride, magnesium fluoride, sodium fluoride, potassium fluoride, stannous fluoride, sodium monofluorophosphate, sodium difluorophosphate, or any combination thereof.

In some aspects, the glass compositions comprise F⁻ in an amount of 0.1 wt. % or more. In some aspects, the glass compositions comprise F⁻ in an amount of 10 wt. % or less. In some aspects, the glass compositions comprise F⁻ in an amount (wt. %) of 0.1-10, 0.1-9.5, 0.1-9, 0.1-8.5, 0.1-8, 0.1-7.5, 0.1-7, 0.1-6.5, 0.1-6, 0.1-5.5, 0.1-5, 0.1-4.5, 0.1-4, 0.1-3.5, 0.1-3, 0.1-2.5, 0.1-2, 0.1-1.5, 0.1-1, 0.1-0.5, 0.5-10, 0.5-9.5, 0.5-9, 0.5-8.5, 0.5-8, 0.5-7.5, 0.5-7, 0.5-6.5, 0.5-6, 0.5-5.5, 0.5-5, 0.5-4.5, 0.5-4, 0.5-3.5, 0.5-3, 0.5-2.5, 0.5-2, 0.5-1.5, 0.5-1, 1-10, 1-9.5, 1-9, 1-8.5, 1-8, 1-7.5, 1-7, 1-6.5, 1-6, 1-5.5, 1-5, 1-4.5, 1-4, 1-3.5, 1-3, 1-2.5, 1-2, 1-1.5, 1.5-10, 1.5-9.5, 1.5-9, 1.5-8.5, 1.5-8, 1.5-7.5, 1.5-7, 1.5-6.5, 1.5-6, 1.5-5.5, 1.5-5, 1.5-4.5, 1.5-4, 1.5-3.5, 1.5-3, 1.5-2.5, 1.5-2, 2-10, 2-9.5, 2-9, 2-8.5, 2-8, 2-7.5, 2-7, 2-6.5, 2-6, 2-5.5, 2-5, 2-4.5, 2-4, 2-3.5, 2-3, 2-2.5, 2.5-10, 2.5-9.5, 2.5-9, 2.5-8.5, 2.5-8, 2.5-7.5, 2.5-7, 2.5-6.5, 2.5-6, 2.5-5.5, 2.5-5, 2.5-4.5, 2.5-4, 2.5-3.5, 2.5-3, 3-10, 3-9.5, 3-9, 3-8.5, 3-8, 3-7.5, 3-7, 3-6.5, 3-6, 3-5.5, 3-5, 3-4.5, 3-4, 3-3.5, 3.5-10, 3.5-9.5, 3.5-9, 3.5-8.5, 3.5-8, 3.5-7.5, 3.5-7, 3.5-6.5, 3.5-6, 3.5-5.5, 3.5-5, 3.5-4.5, 3.5-4, 4-10, 4-9.5, 4-9, 4-8.5, 4-8, 4-7.5, 4-7, 4-6.5, 4-6, 4-5.5, 4-5, 4-4.5, 4.5-10, 4.5-9.5, 4.5-9, 4.5-8.5, 4.5-8, 4.5-7.5, 4.5-7, 4.5-6.5, 4.5-6, 4.5-5.5, 4.5-5, 5-10, 5-9.5, 5-9, 5-8.5, 5-8, 5-7.5, 5-7, 5-6.5, 5-6, 5-5.5, 5.5-10, 5.5-9.5, 5.5-9, 5.5-8.5, 5.5-8, 5.5-7.5, 5.5-7, 5.5-6.5, 5.5-6, 6-10, 6-9.5, 6-9, 6-8.5, 6-8, 6-7.5, 6-7, 6-6.5, 6.5-10, 6.5-9.5, 6.5-9, 6.5-8.5, 6.5-8, 6.5-7.5, 6.5-7, 7-10, 7-9.5, 7-9, 7-8.5, 7-8, 7-7.5, 7.5-10, 7.5-9.5, 7.5-9, 7.5-8.5, 7.5-8, 8-10, 8-9.5, 8-9, 8-8.5, 8.5-10, 8.5-9.5, 8.5-9, 9-10, 9-9.5, or 9.5-10.

Divalent cation oxides (such as alkaline earth oxides and ZnO) improve the melting behavior, chemical durability, and bioactivity of the glass. In addition, alkaline earth oxides may improve other desirable properties in the materials, including influencing the Young's modulus and the coefficient of thermal expansion. In examples, the glass compositions comprise from 30-70 wt. % MO, wherein MO is the sum of MgO, CaO, SrO, BeO, and BaO. In some aspects, the glass compositions comprise MO in an amount (wt. %) of 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 55-70, 55-65, 55-60, 60-70, 60-65, or 65-70.

In some aspects, the glass compositions comprise CaO in an amount of at least 30 wt. %. In some aspects, the glass compositions comprise CaO in an amount of 60 wt. % or less. In some aspects, the glass compositions comprise CaO in an amount (wt. %) of 30-60, 30-55, 30-50, 30-45, 30-40, 30-37, 30-35, 35-60, 35-55, 35-50, 35-45, 35-40, 35-37, 37-60, 37-55, 37-50, 37-45, 37-40, 40-60, 40-55, 40-50, 40-45, 45-60, 45-55, 45-50, 50-60, 50-55, or 55-60.

In some aspects, the glass compositions comprise MgO. In some aspects, the inclusion of MgO can improve liquidus of the precursor glass to avoid devitrification during forming. In some aspects, the glass compositions comprise MgO in an amount of 0 wt. % or greater than 0 wt. %. In some aspects, the glass compositions comprise MgO in an amount of 25 wt. % or less. In some aspects, the glass compositions comprise MgO in an amount (wt. %) of 0-25, 0-20, 0-15, 0-10, 0-5, >0-25, >0-20, >0-15, >0-10, >0-5, 5-25, 5-20, 5-15, 5-10, 10-25, 10-20, 10-15, 15-25, 15-20, or 20-25.

In some aspects, the glass compositions comprise SrO. In some aspects, the inclusion of SrO can improve liquidus of the precursor glass to avoid devitrification during forming. Moreover, in some aspects, SrO may also enter the structure of apatite to improve bioactivity. In some aspects, the glass compositions comprise SrO in an amount of 0 wt. % or greater than 0 wt. %. In some aspects, the glass compositions comprise SrO in an amount of 10 wt. % or less. In some aspects, the glass compositions comprise SrO in an amount (wt. %) of 0-10, 0-9, 0-8, 0-7, 0-6, 0-5, 0-4, 0-3, 0-2, 0-1, 0-0.5, 0-0.1, >0-10, >0-9, >0-8, >0-7, >0-6, >0-5, >0-4, >0-3, >0-2, >0-1, >0-0.5, >0-0.1, 0.1-10, 0.1-9, 0.1-8, 0.1-7, 0.1-6, 0.1-5, 0.1-4, 0.1-3, 0.1-2, 0.1-1, 0.1-0.5, 0.5-10, 0.5-9, 0.5-8, 0.5-7, 0.5-6, 0.5-5, 0.5-4, 0.5-3, 0.5-2, 0.5-1, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10.

In some aspects, the glass compositions comprise ZnO. In some aspects, the inclusion of ZnO can improve liquidus of the precursor glass to avoid devitrification during forming. Moreover, in some aspects, ZnO may also enter the structure of apatite to improve bioactivity. In some aspects, the glass compositions comprise ZnO in an amount of 0 wt. % or greater than 0 wt. %. In some aspects, the glass compositions comprise ZnO in an amount of 10 wt. % or less. In some aspects, the glass compositions comprise ZnO in an amount (wt. %) of 0-10, 0-8, 0-6, 0-4, 0-2, >0-10, >0-8, >0-6, >0-4, >0-2, 2-10, 2-8, 2-6, 2-4, 4-10, 4-8, 4-6, 6-10, 6-8, or 8-10.

In some aspects, CaO is found to be able to react with P₂O₅ to form apatite, and the release of Ca²⁺ ions from the surface of the glass contributes to the formation of a layer rich in calcium phosphate. Thus, the combination of P₂O₅ and CaO may provide advantageous compositions for bioactive glasses.

In some aspects, the glass compositions comprise the sum P₂O₅+CaO in an amount of 40 wt. % or more. In some aspects, the glass compositions comprise the sum P₂O₅+CaO in an amount of 70 wt. % or less. In some aspects, the glass compositions comprise the sum P₂O₅+CaO in an amount (wt. %) of 40-70, 40-65, 40-60, 40-55, 40-50, 40-45, 45-70, 45-65, 45-60, 45-55, 45-50, 50-70, 50-65, 50-60, 50-55, 55-70, 55-65, 55-60, 60-70, 60-65, or 65-70.

In some aspects, the glass compositions comprise the sum MgO+CaO in an amount of at least 30 wt. %. In some aspects, the glass compositions comprise the sum MgO+CaO in an amount of 70 wt. % or less. In some aspects, the glass compositions comprise the sum MgO+CaO in an amount (wt. %) of 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 55-70, 55-65, 55-60, 60-70, 60-65, or 65-70.

In some aspects, the glass compositions comprise the sum F⁻+P₂O₅ in an amount of at least 5 wt. %. In some aspects, the glass compositions comprise the sum F⁻+P₂O₅ in an amount of 30 wt. % or less. In some aspects, the glass compositions comprise the sum F⁻+P₂O₅ in an amount (wt. %) of 5-30, 5-25, 5-20, 5-15, 5-10, 10-30, 10-25, 10-20, 10-15, 15-30, 15-25, 15-20, 20-30, 20-25, or 25-30.

In some aspects, the glass compositions comprise B₂O₃ in an amount of 0 wt. % or greater than 0 wt. %. In some aspects, the glass compositions comprise B₂O₃ in an amount of 5 wt. % or less. In some aspects, the glass compositions comprise B₂O₃ in an amount (wt. %) of 0-5, 0-4.5, 0-4, 0-3.5, 0-3, 0-2.5, 0-2, 0-1.5, 0-1, 0-0.5, 0-0.4, 0-0.3, 0-0.2, 0-0.1, 0-0.05, >0-5, >0-4.5, >0-4, >0-3.5, >0-3, >0-2.5, >0-2, >0-1.5, >0-1, >0-0.5, >0-0.4, >0-0.3, >0-0.2, >0-0.1, >0-0.05, 0.05-5, 0.05-4.5, 0.05-4, 0.05-3.5, 0.05-3, 0.05-2.5, 0.05-2, 0.05-1.5, 0.05-1, 0.05-0.5, 0.05-0.4, 0.05-0.3, 0.05-0.2, 0.05-0.1, 0.1-5, 0.1-4.5, 0.1-4, 0.1-3.5, 0.1-3, 0.1-2.5, 0.1-2, 0.1-1.5, 0.1-1, 0.1-0.5, 0.1-0.4, 0.1-0.3, 0.1-0.2, 0.2-5, 0.2-4.5, 0.2-4, 0.2-3.5, 0.2-3, 0.2-2.5, 0.2-2, 0.2-1.5, 0.2-1, 0.2-0.5, 0.2-0.4, 0.2-0.3, 0.3-5, 0.3-4.5, 0.3-4, 0.3-3.5, 0.3-3, 0.3-2.5, 0.3-2, 0.3-1.5, 0.3-1, 0.3-0.5, 0.3-0.4, 0.4-5, 0.4-4.5, 0.4-4, 0.4-3.5, 0.4-3, 0.4-2.5, 0.4-2, 0.4-1.5, 0.4-1, 0.4-0.5, 0.5-5, 0.5-4.5, 0.5-4, 0.5-3.5, 0.5-3, 0.5-2.5, 0.5-2, 0.5-1.5, 0.5-1, 1-5, 1-4.5, 1-4, 1-3.5, 1-3, 1-2.5, 1-2, 1-1.5, 1.5-5, 1.5-4.5, 1.5-4, 1.5-3.5, 1.5-3, 1.5-2.5, 1.5-2, 2-5, 2-4.5, 2-4, 2-3.5, 2-3, 2-2.5, 2.5-5, 2.5-4.5, 2.5-4, 2.5-3.5, 2.5-3, 3-5, 3-4.5, 3-4, 3-3.5, 3.5-5, 3.5-4.5, 3.5-4, 4-5, 4-4.5, or 4.5-5.

In some aspects, the glass compositions can comprise Al₂O₃. In some aspects, Al₂O₃ may influence (e.g., stabilize) the network structure of the glass, improve mechanical properties, improve chemical durability, or any combination thereof. In some aspects, Al₂O₃ may lower liquidus temperature, lower coefficient of thermal expansion, enhance the strain point, or any combination thereof. In some aspects, in addition to its role as a network former, Al₂O₃ (and ZrO₂) may help improve the chemical durability and mechanical properties in silicate glass while having no toxicity concerns. In some aspects, too high a content of Al₂O₃ or ZrO₂ (e.g., >20 wt. %) generally increases the viscosity of the melt and decreases bioactivity.

In some aspects, the glass compositions comprise Al₂O₃ in an amount of 0 wt. % or greater than 0 wt. %. In some aspects, the glass compositions comprise Al₂O₃ in an amount of 5 wt. % or less. In some aspects, the glass compositions comprise Al₂O₃ in an amount (wt. %) of 0-5, 0-4.5, 0-4, 0-3.5, 0-3, 0-2.5, 0-2, 0-1.5, 0-1, 0-0.5, 0-0.4, 0-0.3, 0-0.2, 0-0.1, 0-0.05, >0-5, >0-4.5, >0-4, >0-3.5, >0-3, >0-2.5, >0-2, >0-1.5, >0-1, >0-0.5, >0-0.4, >0-0.3, >0-0.2, >0-0.1, >0-0.05, 0.05-5, 0.05-4.5, 0.05-4, 0.05-3.5, 0.05-3, 0.05-2.5, 0.05-2, 0.05-1.5, 0.05-1, 0.05-0.5, 0.05-0.4, 0.05-0.3, 0.05-0.2, 0.05-0.1, 0.1-5, 0.1-4.5, 0.1-4, 0.1-3.5, 0.1-3, 0.1-2.5, 0.1-2, 0.1-1.5, 0.1-1, 0.1-0.5, 0.1-0.4, 0.1-0.3, 0.1-0.2, 0.2-5, 0.2-4.5, 0.2-4, 0.2-3.5, 0.2-3, 0.2-2.5, 0.2-2, 0.2-1.5, 0.2-1, 0.2-0.5, 0.2-0.4, 0.2-0.3, 0.3-5, 0.3-4.5, 0.3-4, 0.3-3.5, 0.3-3, 0.3-2.5, 0.3-2, 0.3-1.5, 0.3-1, 0.3-0.5, 0.3-0.4, 0.4-5, 0.4-4.5, 0.4-4, 0.4-3.5, 0.4-3, 0.4-2.5, 0.4-2, 0.4-1.5, 0.4-1, 0.4-0.5, 0.5-5, 0.5-4.5, 0.5-4, 0.5-3.5, 0.5-3, 0.5-2.5, 0.5-2, 0.5-1.5, 0.5-1, 1-5, 1-4.5, 1-4, 1-3.5, 1-3, 1-2.5, 1-2, 1-1.5, 1.5-5, 1.5-4.5, 1.5-4, 1.5-3.5, 1.5-3, 1.5-2.5, 1.5-2, 2-5, 2-4.5, 2-4, 2-3.5, 2-3, 2-2.5, 2.5-5, 2.5-4.5, 2.5-4, 2.5-3.5, 2.5-3, 3-5, 3-4.5, 3-4, 3-3.5, 3.5-5, 3.5-4.5, 3.5-4, 4-5, 4-4.5, or 4.5-5.

In some aspects, alkali oxides (Na₂O, K₂O, Li₂O, Rb₂O, or Cs₂O) serve as aids in achieving low melting temperature and low liquidus temperatures, which can aid glass melting processes. In addition, in some aspects, the addition of alkali oxides can improve bioactivity. However, in some aspects, if the amount of alkali oxides is too high, the glass compositions have reduced chemical durability; in other words, in some aspects, keeping the amount of alkali oxides to a certain lower range achieves high chemical durability of the glass compositions. In some aspects, the glass compositions do not contain any alkali oxides (e.g., do not contain any added alkali oxides). In some aspects, the glass compositions are substantially free of alkali oxides (e.g., contain less than 0.1 wt. % alkali oxides).

In some aspects, the glass compositions comprise Li₂O in an amount of 0 wt. % or greater than 0 wt. %. In some aspects, the glass compositions comprise Li₂O in an amount of 10 wt. % or less. In some aspects, the glass compositions comprise Li₂O in an amount (wt. %) of 0-10, 0-9, 0-8, 0-7, 0-6, 0-5, 0-4, 0-3, 0-2, 0-1, 0-0.5, 0-0.1, >0-10, >0-9, >0-8, >0-7, >0-6, >0-5, >0-4, >0-3, >0-2, >0-1, >0-0.5, >0-0.1, 0.1-10, 0.1-9, 0.1-8, 0.1-7, 0.1-6, 0.1-5, 0.1-4, 0.1-3, 0.1-2, 0.1-1, 0.1-0.5, 0.5-10, 0.5-9, 0.5-8, 0.5-7, 0.5-6, 0.5-5, 0.5-4, 0.5-3, 0.5-2, 0.5-1, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10.

In some aspects, the glass compositions comprise Na₂O in an amount of 0 wt. % or greater than 0 wt. %. In some aspects, the glass compositions comprise Na₂O in an amount of 10 wt. % or less. In some aspects, the glass compositions comprise Na₂O in an amount (wt. %) of 0-10, 0-9, 0-8, 0-7, 0-6, 0-5, 0-4, 0-3, 0-2, 0-1, 0-0.5, 0-0.1, >0-10, >0-9, >0-8, >0-7, >0-6, >0-5, >0-4, >0-3, >0-2, >0-1, >0-0.5, >0-0.1, 0.1-10, 0.1-9, 0.1-8, 0.1-7, 0.1-6, 0.1-5, 0.1-4, 0.1-3, 0.1-2, 0.1-1, 0.1-0.5, 0.5-10, 0.5-9, 0.5-8, 0.5-7, 0.5-6, 0.5-5, 0.5-4, 0.5-3, 0.5-2, 0.5-1, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10.

In some aspects, the glass compositions comprise K₂O in an amount of 0 wt. % or greater than 0 wt. %. In some aspects, the glass compositions comprise K₂O in an amount of 10 wt. % or less. In some aspects, the glass compositions comprise K₂O in an amount (wt. %) of 0-10, 0-9, 0-8, 0-7, 0-6, 0-5, 0-4, 0-3, 0-2, 0-1, 0-0.5, 0-0.1, >0-10, >0-9, >0-8, >0-7, >0-6, >0-5, >0-4, >0-3, >0-2, >0-1, >0-0.5, >0-0.1, 0.1-10, 0.1-9, 0.1-8, 0.1-7, 0.1-6, 0.1-5, 0.1-4, 0.1-3, 0.1-2, 0.1-1, 0.1-0.5, 0.5-10, 0.5-9, 0.5-8, 0.5-7, 0.5-6, 0.5-5, 0.5-4, 0.5-3, 0.5-2, 0.5-1, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10.

In some aspects, the glass compositions comprise the sum Li₂O+Na₂O+K₂O in an amount of 0 wt. % or greater than 0 wt. %. In some aspects, the glass compositions comprise the sum Li₂O+Na₂O+K₂O in an amount of 10 wt. % or less, 1 wt. % or less, or 0.5 wt. % or less. In some aspects, the glass compositions comprise the sum Li₂O+Na₂O+K₂O in an amount (wt. %) of 0-10, 0-9, 0-8, 0-7, 0-6, 0-5, 0-4, 0-3, 0-2, 0-1, 0-0.5, 0-0.1, >0-10, >0-9, >0-8, >0-7, >0-6, >0-5, >0-4, >0-3, >0-2, >0-1, >0-0.5, >0-0.1, 0.1-10, 0.1-9, 0.1-8, 0.1-7, 0.1-6, 0.1-5, 0.1-4, 0.1-3, 0.1-2, 0.1-1, 0.1-0.5, 0.5-10, 0.5-9, 0.5-8, 0.5-7, 0.5-6, 0.5-5, 0.5-4, 0.5-3, 0.5-2, 0.5-1, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10. In some aspects, the glass compositions comprise the sum Na₂O+K₂O+Li₂O+Rb₂O+Cs₂O in any of the amounts set forth in this paragraph.

In some aspects, additional components can be incorporated into the glass compositions to provide additional benefits or may be incorporated as contaminants typically found in commercially-prepared glass. For example, additional components can be added as coloring or fining agents (e.g., to facilitate removal of gaseous inclusions from melted batch materials used to produce the glass compositions) and/or for other purposes. In some aspects, the glass compositions may comprise one or more compounds useful as ultraviolet radiation absorbers. In some aspects, the glass compositions can comprise 3 wt. % or less ZnO, TiO₂, CeO, MnO, Nb₂O₅, MoO₃, Ta₂O₅, WO₃, SnO₂, Fe₂O₃, As₂O₃, Sb₂O₃, Cl, Br, or any combination thereof. In some aspects, the glass compositions can comprise from 0 to about 3 wt. %, 0 to about 2 wt. %, 0 to about 1 wt. %, 0 to 0.5 wt. %, 0 to 0.1 wt. %, 0 to 0.05 wt. %, or 0 to 0.01 wt. % ZnO, TiO₂, CeO, MnO, Nb₂O₅, MoO₃, Ta₂O₅, WO₃, SnO₂, Fe₂O₃, As₂O₃, Sb₂O₃, Cl, Br, or any combination thereof. In some aspects, the glass compositions can also include various contaminants associated with batch materials and/or introduced into the glass by the melting, fining, and/or forming equipment used to produce the glass. For example, in some aspects, the glass compositions can comprise from 0 to about 3 wt. %, 0 to about 2 wt. %, 0 to about 1 wt. %, 0 to about 0.5 wt. %, 0 to about 0.1 wt. %, 0 to about 0.05 wt. %, or 0 to about 0.01 wt. % SnO₂ or Fe₂O₃, or a combination thereof.

In some aspects, the glass composition is a glass-ceramic (e.g., a glass-ceramic article). In some aspects, the glass composition contains a crystalline phase. In some aspects, the crystalline phase is or comprises fluorapatite. In some aspects, the crystalline phase is or comprises fluorapatite, calcium silicate, calcium phosphate, calcium fluoride, or any combination thereof. In some aspects, the glass composition is a glass-ceramic article comprising a fluorapatite crystal phase. In some aspects, the fluorapatite crystal phase is the major crystalline phase (e.g., more than 50 wt. %). In some aspects, the fluorapatite phase is present in the glass composition in an amount (wt. %) of >50-95, >50-90, >50-85, >50-80, >50-75, >50-70, >50-65, >50-60, >50-55, 55-95, 55-90, 55-85, 55-80, 55-75, 55-70, 55-65, 55-60, 60-95, 60-90, 60-85, 60-80, 60-75, 60-70, 60-65, 65-95, 65-90, 65-85, 65-80, 65-75, 65-70, 70-95, 70-90, 70-85, 70-80, 70-75, 75-95, 75-90, 75-85, 75-80, 80-95, 80-90, 80-85, 85-95, 85-90, or 90-95. Amount of a given crystal phase can be measured by routine XRD analysis.

In some aspects, the glass composition comprises at least one minor crystalline phase of calcium silicate, calcium phosphate, calcium fluoride, or any combination thereof. In some aspects, the amount (wt. %) of each minor crystalline phase separately, or the amount (wt. %) of the sum total of all minor crystal phases, can be described by any of the following amounts: 1-<50, 1-49, 1-45, 1-40, 1-35, 1-30, 1-25, 1-20, 1-15, 1-10, 1-9, 1-8, 1-7, 1-6, 1-5, 1-4, 1-3, 1-2, 2-<50, 2-49, 2-45, 2-40, 2-35, 2-30, 2-25, 2-20, 2-15, 2-10, 2-9, 2-8, 2-7, 2-6, 2-5, 2-4, 2-3, 3-<50, 3-49, 3-45, 3-40, 3-35, 3-30, 3-25, 3-20, 3-15, 3-10, 3-9, 3-8, 3-7, 3-6, 3-5, 3-4, 4-<50, 4-49, 4-45, 4-40, 4-35, 4-30, 4-25, 4-20, 4-15, 4-10, 4-9, 4-8, 4-7, 4-6, 4-5, 5-<50, 5-49, 5-45, 5-40, 5-35, 5-30, 5-25, 5-20, 5-15, 5-10, 5-9, 5-8, 5-7, 5-6, 6-<50, 6-49, 6-45, 6-40, 6-35, 6-30, 6-25, 6-20, 6-15, 6-10, 6-9, 6-8, 6-7, 7-<50, 7-49, 7-45, 7-40, 7-35, 7-30, 7-25, 7-20, 7-15, 7-10, 7-9, 7-8, 8-<50, 8-49, 8-45, 8-40, 8-35, 8-30, 8-25, 8-20, 8-15, 8-10, 8-9, 9-<50, 9-49, 9-45, 9-40, 9-35, 9-30, 9-25, 9-20, 9-15, 9-10, 10-<50, 10-49, 10-45, 10-40, 10-35, 10-30, 10-25, 10-20, 10-15, 15-<50, 15-49, 15-45, 15-40, 15-35, 15-30, 15-25, 15-20, 20-<50, 20-49, 20-45, 20-40, 20-35, 20-30, 20-25, 25-<50, 25-49, 25-45, 25-40, 25-35, 25-30, 30-<50, 30-49, 30-45, 30-40, 30-35, 35-<50, 35-49, 35-45, 35-40, 40-<50, 40-49, 40-45, 45-<50, 45-49, or 49-<50. Amount of a given crystal phase can be measured by routine XRD analysis.

In some aspects, the glass composition is a glass-ceramic article comprising a glass phase. In some aspects, the glass-ceramic article comprises a glass phase in an amount of at least 5 wt. %. In some aspects, the glass-ceramic article comprises a glass phase in an amount of 95 wt. % or less. In some aspects, the glass-ceramic article comprises a glass phase in an amount of 5-95, 5-90, 5-85, 5-80, 5-75, 5-70, 5-65, 5-60, 5-55, 5-50, 5-45, 5-40, 5-35, 5-30, 5-25, 5-20, 5-15, 5-10, 10-95, 10-90, 10-85, 10-80, 10-75, 10-70, 10-65, 10-60, 10-55, 10-50, 10-45, 10-40, 10-35, 10-30, 10-25, 10-20, 10-15, 15-95, 15-90, 15-85, 15-80, 15-75, 15-70, 15-65, 15-60, 15-55, 15-50, 15-45, 15-40, 15-35, 15-30, 15-25, 15-20, 20-95, 20-90, 20-85, 20-80, 20-75, 20-70, 20-65, 20-60, 20-55, 20-50, 20-45, 20-40, 20-35, 20-30, 20-25, 25-95, 25-90, 25-85, 25-80, 25-75, 25-70, 25-65, 25-60, 25-55, 25-50, 25-45, 25-40, 25-35, 25-30, 30-95, 30-90, 30-85, 30-80, 30-75, 30-70, 30-65, 30-60, 30-55, 30-50, 30-45, 30-40, 30-35, 35-95, 35-90, 35-85, 35-0, 35-75, 35-70, 35-65, 35-60, 35-55, 35-50, 35-45, 35-40, 40-95, 40-90, 40-85, 40-80, 40-75, 40-70, 40-65, 40-60, 40-55, 40-50, 40-45, 45-95, 45-90, 45-5, 45-0, 45-5, 45-0, 45-65, 45-60, 45-55, 45-50, 50-95, 50-90, 50-5, 50-0, 50-75, 50-0, 50-65, 50-60, 50-55, 55-95, 55-90, 55-85, 55-80, 55-75, 55-70, 55-65, 55-60, 60-95, 60-90, 60-85, 60-80, 60-75, 60-70, 60-65, 65-95, 65-90, 65-85, 65-80, 65-75, 65-70, 70-95, 70-90, 70-85, 70-80, 70-75, 75-95, 75-90, 75-85, 75-80, 80-95, 80-90, 80-85, 85-95, 85-90, or 90-95. Amount of glass phase can be measured by routine XRD analysis.

In some aspects, the glass composition is a glass-ceramic and comprises a total crystallinity of at least 5 wt. %. In some aspects, the glass composition is a glass-ceramic and comprises a total crystallinity of 50 wt. % or less. In some aspects, the glass composition is a glass-ceramic and comprises a total crystallinity (wt. %) of 5-50, 5-45, 5-40, 5-35, 5-30, 5-25, 5-20, 5-15, 5-10, 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45, or 45-50. Total crystallinity can be measured by routine XRD analysis.

In some aspects, the glass composition is substantially free of, or does not contain, a crystalline phase. For example, in some aspects, the glass composition has a glassy phase and substantially no crystalline phase. In some aspects, the glass composition is a glass (e.g., a glass article).

In some aspects, the glass composition is in a form of particulates, microbeads, fibers, or a combination thereof. In some aspects, the particulates are a fine powder. Methods for preparing particulates, microbeads, and/or fibers from glass compositions is known in the art. In some aspects, the particulates, microbeads, and/or fibers have an average particle size of at least 1 micron. In some aspects, the particulates and/or microbeads have an average particle size of 50 microns or less. In some aspects, the particulates and/or microbeads have an average particle size (microns) of 1-50, 1-45, 1-40, 1-35, 1-30, 1-25, 1-20, 1-15, 1-10, 1-5, 5-50, 5-45, 5-40, 5-35, 5-30, 5-25, 5-20, 5-15, 5-10, 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45, or 45-50. Average particle size, as used herein, means the average size as measured by dynamic light scattering using commercially available equipment.

In some aspects, the glass compositions can be made via traditional methods; however, in some aspects, the glass composition is not subjected to a subsequent heat treatment after melting and cooling, which subsequent heat treatment is normally necessary for prior compositions to provide a crystalline phase. In contrast, for the glass compositions disclosed herein, after melting precursor components of a glass composition, upon cooling one or more crystalline phases is spontaneously formed without a need for a subsequent heat treatment. For example, in some aspects, precursor glasses can be formed by thoroughly mixing the requisite batch materials (for example, using a Turbula® mixer) in order to secure a homogeneous melt, and subsequently placing into silica and/or platinum crucibles. The crucibles can be placed into a furnace and the glass batch melted and maintained at temperatures ranging from 1100° C. to 1400° C. for times ranging from about 6 hours to 24 hours. The melts can thereafter be poured into steel molds to yield glass slabs. Upon cooling, one or more crystalline phases (e.g., comprising fluorapatite) spontaneously form. However, in some aspects, subsequently, those slabs optionally can be transferred immediately to an annealer operating at about 400° C. to 700° C., where the glass is held at temperature for about 0.5 hour to 3 hours and subsequently cooled overnight so as to tune the amount and type of crystalline phase(s) present. In another non-limiting example, precursor glasses are prepared by dry blending the appropriate oxides and mineral sources for a time sufficient to thoroughly mix the ingredients. The glasses are melted in platinum crucibles at temperatures ranging from about 1100° C. to 1400° C. and held at temperature for about 6 hours to 16 hours. The resulting glass melts are then poured onto a steel table to cool, spontaneously resulting in one or more crystalline phases without the need to subject the cooled mixture to a subsequent thermal treatment. However, if desired, the cooled mixture optionally is then annealed at appropriate temperatures so as to tune the amount and type of crystalline phase(s).

In some aspects, the resulting glass compositions can be ground into fine particles in the range of 1-10 microns (m) by air jet milling. The particle size can be varied in the range of 1-100 m using attrition milling or ball milling of glass frits. Furthermore, these glasses can be processed into short fibers, beads, sheets or three-dimensional scaffolds using different methods. Short fibers are made by melt spinning or electric spinning; beads can be produced by flowing glass particles through a hot vertical furnace or a flame torch; sheets can be manufactured using thin rolling, float or fusion-draw processes; and scaffolds can be produced using rapid prototyping, polymer foam replication and particle sintering. Glasses of desired forms can be used to support cell growth, soft and hard tissue regeneration, stimulation of gene expression or angiogenesis.

Continuous fibers can be easily drawn from the claimed composition using processes known in the art. For example, fibers can be formed using a directly heated (electricity passing directly through) platinum bushing. Glass cullet is loaded into the bushing, heated up until the glass can melt. Temperatures are set to achieve a desired glass viscosity (usually <1000 poise) allowing a drip to form on the orifice in the bushing (Bushing size is selected to create a restriction that influences possible fiber diameter ranges). The drip is pulled by hand to begin forming a fiber. Once a fiber is established it is connected to a rotating pulling/collection drum to continue the pulling process at a consistent speed. Using the drum speed (or revolutions per minute RPM) and glass viscosity the fiber diameter can be manipulated—in general the faster the pull speed, the smaller the fiber diameter. Glass fibers with diameters in the range of 1-100 μm can be drawn continuously from a glass melt. Fibers can also be created using an updraw process. In this process, fibers are pulled from a glass melt surface sitting in a box furnace. By controlling the viscosity of the glass, a quartz rod is used to pull glass from the melt surface to form a fiber. The fiber can be continuously pulled upward to increase the fiber length. The velocity that the rod is pulled up determines the fiber thickness along with the viscosity of the glass.

Therefore, in some aspects, a method for making the glass compositions disclosed herein is provided, the method comprising:
combining each component of the glass composition to form a mixture,
heating the mixture to a temperature of 1500° C. or less to form a melted mixture, and
cooling the melted mixture to form a cooled mixture.

In some aspects, the heating step employs any suitable temperature. In some aspects, the heating step employs a temperature sufficient to melt the components to form a mixture. In some aspects, the temperature (° C.) is 1000-1500, 1000-1450, 1000-1400, 1000-1350, 1000-1300, 1000-1250, 1000-1200, 1000-1150, 1000-1100, 1000-1050, 1050-1500, 1050-1450, 1050-1400, 1050-1350, 1050-1300, 1050-1250, 1050-1200, 1050-1150, 1050-1100, 1100-1500, 1100-1450, 1100-1400, 1100-1350, 1100-1300, 1100-1250, 1100-1200, 1100-1150, 1150-1500, 1150-1450, 1150-1400, 1150-1350, 1150-1300, 1150-1250, 1150-1200, 1200-1500, 1200-1450, 1200-1400, 1200-1350, 1200-1300, 1200-1250, 1250-1500, 1250-1450, 1250-1400, 1250-1350, 1250-1300, 1300-1500, 1300-1450, 1300-1400, 1300-1350, 1350-1500, 1350-1450, 1350-1400, 1400-1500, 1400-1450, or 1450-1500.

In some aspects, the heating step is employed for any suitable time period. In some aspects, the heating step employs a time period sufficient to melt the components to form a mixture. In some aspects, the time period is at least 6 hours. In some aspects, the time period is 30 hours or less. In some aspects, the time period (hours) is 6-30, 6-28, 6-26, 6-24, 6-22, 6-20, 6-18, 6-16, 6-14, 6-12, 6-10, 6-8, 8-30, 8-28, 8-26, 8-24, 8-22, 8-20, 8-18, 8-16, 8-14, 8-12, 8-10, 10-30, 10-28, 10-26, 10-24, 10-22, 10-20, 10-18, 10-16, 10-14, 10-12, 12-30, 12-28, 12-26, 12-24, 12-22, 12-20, 12-18, 12-16, 12-14, 14-30, 14-28, 14-26, 14-24, 14-22, 14-20, 14-18, 14-16, 16-30, 16-28, 16-26, 16-24, 16-22, 16-20, 16-18, 18-30,18-28, 18-26, 18-24, 18-22, 18-20, 20-30, 20-28, 20-26, 20-24, 20-22, 22-30, 22-28, 22-26, 22-24, 24-30, 24-28, 24-26, 26-30, 26-28, or 28-30.

In some aspects, the cooling step spontaneously results in at least partial crystallization into a fluorapatite crystal phase. In some aspects, In some aspects, the cooling step spontaneously results in at least partial crystallization into one or more crystalline phases, such as fluorapatite, calcium silicate, calcium phosphate, calcium fluoride, or any combination thereof. In some aspects, the fluorapatite crystal phase is the major crystal phase. In some aspects, calcium silicate, calcium phosphate, and/or calcium fluoride are minor crystalline phases, taken alone or in combination.

In some aspects, after the cooling step, the cooled mixture is not heated to a temperature sufficient to result in crystallization. In some aspects, after the cooling step, the cooled mixture is not heated to a crystallization temperature. In some aspects, the crystallization temperature is 500-1000° C. depending on the composition. In some aspects, the crystallization temperature (° C.) is 500-1000, 500-950, 500-900, 500-850, 500-800, 500-750, 500-700, 500-650, 500-600, 500-550, 550-1000, 550-950, 550-900, 550-850, 550-800, 550-750, 550-700, 550-650, 550-600, 600-1000, 600-950, 600-900, 600-850, 600-800, 600-750, 600-700, 600-650, 650-1000, 650-950, 650-900, 650-850, 650-800, 650-750, 650-700, 700-1000, 700-950, 700-900, 700-850, 700-800, 700-750, 750-1000, 750-950, 750-900, 750-850, 750-800, 800-1000, 800-950, 800-900, 800-850, 850-1000, 850-950, 850-900, 900-1000, 900-950, or 950-1000.

In some aspects, the method further comprising forming the cooled mixture into particulates, microbeads, fibers, or a combination thereof.

Aspects are related to compositions or matrices containing glass compositions and the methods of using the matrices to treat medical conditions. The matrices can be a toothpaste, mouthwash, gel, varnish, rinse, spray, ointment, salve, cream, bandage, polymer film, oral formulation, pill, capsule, transdermal formulation, or the like. The glass compositions claimed can be physically or chemically attached to matrices or other matrix components, or simply mixed in. As noted above, the glass composition can be in the form of a bioactive glass that can be in any form that works in the application, including particles, beads, particulates, short fibers, long fibers, or woolen meshes. The methods of using the glass-containing matrices to treat a medical condition can be the use of matrix as normally applied or used.

In some aspects, disclosed is a matrix comprising a glass composition. In some aspects, the matrix comprises a glass composition, wherein the glass composition is attached to the matrix or mixed therein. In some aspects, the matrix includes at least one of: a toothpaste, mouthwash, gel, varnish, rinse, spray, ointment, salve, cream, bandage, polymer film, oral formulation, pill, capsule, or transdermal formulation.

In some aspects, the matrix is substantially free of, or does not contain, a fluoride ion source that is derived from the glass composition (e.g., a fluoride ion source that is added to the matrix as a component separate from the glass composition and which fluoride ion source is not derived from the glass composition). In this regard, in some aspects, the matrix is substantially free of, or does not contain, calcium fluoride, magnesium fluoride, sodium fluoride, potassium fluoride, stannous fluoride, sodium monofluorophosphate, sodium difluorophosphate, or any combination thereof. In some aspects, the matrix is substantially free of, or does not contain, a source of fluoride ions that is added to the matrix separately from the glass composition. In some aspects, the matrix includes a fluoride ion source that is added separately to the matrix. In some aspects, the matrix comprises calcium fluoride, magnesium fluoride, sodium fluoride, potassium fluoride, stannous fluoride, sodium monofluorophosphate, sodium difluorophosphate, or any combination thereof. In some aspects, the calcium fluoride, magnesium fluoride, sodium fluoride, potassium fluoride, stannous fluoride, sodium monofluorophosphate, sodium difluorophosphate, or any combination thereof are added to the matrix separately from the glass composition.

In some aspects, the matrix is or comprises a dentifrice composition. In some aspects, the dentifrice composition is a toothpaste, a mouthwash, or a gel, or any other formulation that is intended for oral care. In some aspects, without wishing to be bound by theory, it is believed that high chemical durability and high bioactivity of the glass compositions makes them advantageous for use in oral care formulations (e.g., dentifrice compositions) or cosmetic formulations.

In some aspects, the dentifrice compositions can comprise any suitable components and amounts of such components. For example, in some aspects, the dentifrice compositions comprise the glass composition (e.g., a glass-ceramic comprising fluorapatite) in combination with one or more of a solvent, a humectant, a fluoride ion source, a flavoring, a surfactant, a sweetener, a whitening agent, an abrasive, or any combination thereof. Such dentifrice compositions may be aqueous based or they may be non-aqueous based. When aqueous based, the solvent comprises water. When non-aqueous based, the solvent typically comprises glycerin. In some aspects, a suitable dentifrice composition with ranges of components is shown in Table 1.

TABLE 1

| Component | Amount (wt. %) |
|---|---|
| Glass Composition | >0-20 |
| Solvent* | 10-80 |
| Humectant | 2-50 |
| Fluoride Ion Source | 0-1 |
| Flavoring | 0-0.5 |
| Surfactant | >0-5 |
| Sweetener | 0-5 |
| Whitening Agent | 0-2 |
| Abrasive | 0-20 |
| Other | 0-10 |

*Solvent can be water, glycerin, or a combination thereof

In some aspects, the dentifrice compositions comprise the glass composition in amount of greater than 0 wt. %. In some aspects, the dentifrice compositions comprise the glass composition in amount of 20 wt. % or less. In some aspects, the dentifrice compositions comprise the glass composition in amount (wt. %) of >0-20, >0-18, >0-16, >0-14, >0-12, >0-10, >0-8, >0-6, >0-4, >0-2, >0-1, >0-0.5, >0-0.1, 0.1-20, 0.1-18, 0.1-16, 0.1-14, 0.1-12, 0.1-10, 0.1-8, 0.1-6, 0.1-4, 0.1-2, 0.1-1, 0.1-0.5, 0.5-20, 0.5-18, 0.5-16, 0.5-14, 0.5-12, 0.5-10, 0.5-8, 0.5-6, 0.5-4, 0.5-2, 0.5-1, 1-20, 1-18, 1-16, 1-14, 1-12, 1-10, 1-8, 1-6, 1-4, 1-2, 2-20, 2-18, 2-6, 2-14, 2-12, 2-10, 2-8, 2-6, 2-4, 4-20, 4-18, 4-16, 4-14, 4-12, 4-10, 4-8, 4-6, 6-20, 6-18, 6-16, 6-14, 6-12, 6-10, 6-8, 8-20, 8-18, 8-16, 8-14, 8-12, 8-10, 10-20, 10-18, 10-16, 10-14, 10-12, 12-20, 12-18, 12-16, 12-14, 14-20, 14-18, 14-16, 16-20, 16-18, or 18-20.

In some aspects, the dentifrice compositions comprise a solvent in an amount of 10 wt. % or more. In some aspects, the dentifrice compositions comprise a solvent in an amount of 80 wt. % or less. In some aspects, the dentifrice compositions comprise a solvent in an amount (wt. %) of 10-80, 10-75, 10-70,10-65, 10-60, 10-55, 10-50, 10-45, 10-40, 10-35, 10-30, 10-25, 10-20, 10-15, 15-80, 15-75, 15-70, 15-65, 15-60, 15-55, 15-50, 15-45, 15-40, 15-35, 15-30, 15-25, 15-20, 20-80, 20-75, 20-70, 20-65, 20-60, 20-55, 20-50, 20-45, 20-40, 20-35, 20-30, 20-25, 25-80, 25-75, 25-70, 25-65, 25-60, 25-55, 25-50, 25-45, 25-40, 25-35, 25-30, 30-80, 30-75, 30-70, 30-65, 30-60, 30-55, 30-50, 30-45, 30-40, 30-35, 35-80, 35-75, 35-70, 35-65, 35-60, 35-55, 35-50, 35-45, 35-40, 40-80, 40-75, 40-70, 40-65, 40-60, 40-55, 40-50, 40-45, 45-80, 45-75, 45-70, 45-65, 45-60, 45-55, 45-50, 50-80, 50-75, 50-70, 50-65, 50-60, 50-55, 55-80, 55-75, 55-70, 55-65, 55-60, 60-80, 60-75, 60-70, 60-65, 65-80, 65-75, 65-70, 70-80, 70-75, or 75-80. As disclosed elsewhere herein, the solvent can be aqueous or non-aqueous. Generally, when aqueous, the solvent is or comprises water, and when non-aqueous, the solvent is or comprises glycerin. In some aspects, a combination of water and glycerin is employed, and the weight percent of glycerin in such a mixture can be 1-99, 5-95, 10-90, 15-85, 20-80, 25-75, 30-70, 35-65, 40-60, 45-55, 1-50, 5-45, 10-40, 15-35, 50-95, 55-90, 60-85, or 65-80.

In some aspects, the dentifrice compositions comprise one or more humectants. In some aspects, the humectants can include short-chained polyalcohols For example, short-chained polyalcohols can include glycerol, sorbitol, propylene glycol, polyethylene glycol, or any combination thereof. In some aspects, the dentifrice compositions comprise one or more humectants in an amount of 2 wt. % or more. In some aspects, the dentifrice compositions comprise one or more humectants in an amount of 50 wt. % or less. In some aspects, the dentifrice compositions comprise one or more humectants in an amount (wt. %) of 2-50, 2-45, 2-40, 2-35, 2-30, 2-25, 2-20, 2-15, 2-10, 2-5, 5-50, 5-45, 5-40, 5-35, 5-30, 5-25, 5-20, 5-15, 5-10, 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45, or 45-50.

In some aspects, the dentifrice composition comprises one or more fluoride ion sources. In some aspects, the one or more fluoride ion sources includes calcium fluoride, magnesium fluoride, sodium fluoride, potassium fluoride, stannous fluoride, sodium monofluorophosphate, sodium difluorophosphate, or any combination thereof. In some aspects, the dentifrice compositions comprise one or more fluoride ion sources in an amount of 0 wt. % or greater than 0 wt. %. In some aspects, the dentifrice compositions comprise one or more fluoride ion sources in an amount of 1 wt. % or less. In some aspects, the dentifrice compositions comprise one or more fluoride ion sources in an amount (wt. %) of 0-1, 0-0.9, 0-0.8, 0-0.7, 0-0.6, 0-0.5, 0-0.4, 0-0.3, 0-0.2, 0-0.1, 0.-0.05, >0-1, >0-0.9, >0-0.8, >0-0.7, >0-0.6, >0-0.5, >0-0.4, >0-0.3, >0-0.2, >0-0.1, >0-0.05, 0.05-1, 0.05-0.9, 0.05-0.8, 0.05-0.7, 0.05-0.6, 0.05-0.5, 0.05-0.4, 0.05-0.3, 0.05-0.2, 0.05-0.1, 0.1-1, 0.1-0.9, 0.1-0.8, 0.1-0.7, 0.1-0.6, 0.1-0.5, 0.1-0.4, 0.1-0.3, 0.1-0.2, 0.2-1, 0.2-0.9, 0.2-0.8, 0.2-0.7, 0.2-0.6, 0.2-0.5, 0.2-0.4, 0.2-0.3, 0.3-1, 0.3-0.9, 0.3-0.8, 0.3-0.7, 0.3-0.6, 0.3-0.5, 0.3-0.4, 0.4-1, 0.4-0.9, 0.4-0.8, 0.4-0.7, 0.4-0.6, 0.4-0.5, 0.5-1, 0.5-0.9, 0.5-0.8, 0.5-0.7, 0.5-0.6, 0.6-1, 0.6-0.9, 0.6-0.8, 0.6-0.7, 0.7-1, 0.7-0.9, 0.7-0.8, 0.8-1, 0.8-0.9, or 0.9-1.

In some aspects, the dentifrice compositions comprise one or more flavoring agents in an amount of 0 wt. % or greater than 0 wt. %. In some aspects, the dentifrice compositions comprise one or more flavoring agents in an amount of 0.5 wt. % or less. In some aspects, the dentifrice compositions comprise one or more flavoring agents in an amount (wt. %) of 0-0.5, 0-0.4, 0-0.3, 0-0.2, 0-0.1, 0-0.05, >0-0.5, >0-0.4, >0-0.3, >0-0.2, >0-0.1, >0-0.05, 0.05-0.5, 0.05-0.4, 0.05-0.3, 0.05-0.2, 0.05-0.1, 0.1-0.5, 0.1-0.4, 0.1-0.3, 0.1-0.2, 0.2-0.5, 0.2-0.4, 0.2-0.3, 0.3-0.5, 0.3-0.4, or 0.4-0.5.

In some aspects, the dentifrice compositions comprise one or more surfactants. In some aspects, the one or more surfactants include sodium lauryl sulfate, poloxamer (such as an ethylene oxide/propylene oxide copolymers), and any combination thereof. In some aspects, sodium lauryl sulfate exacerbates tooth sensitivity issues and therefore in some aspects sodium lauryl sulfate is not included in the dentifrice compositions. In some aspects, the dentifrice compositions comprise one or more surfactants in an amount of greater than 0 wt. %. In some aspects, the dentifrice compositions comprise one or more surfactants in an amount of 5 wt. % or less. In some aspects, the dentifrice compositions comprise one or more surfactants in an amount (wt. %) of >0-5, >0-4.5, >0-4, >0-3.5, >0-3, >0-2.5, >0-2, >0-1.5, >0-1, >0-0.5, >0-0.1, 0.1-5, 0.1-4.5, 0.1-4, 0.1-3.5, 0.1-3, 0.1-2.5, 0.1-2, 0.1-1.5, 0.1-1, 0.1-0.5, 0.5-5, 0.5-4.5, 0.5-4, 0.5-3.5, 0.5-3, 0.5-2.5, 0.5-2, 0.5-1.5, 0.5-1, 1-5, 1-4.5, 1-4, 1-3.5, 1-3, 1-2.5, 1-2, 1-1.5, 1.5-5, 1.5-4.5, 1.5-4, 1.5-3.5, 1.5-3, 1.5-2.5, 1.5-2, 2-5, 2-4.5, 2-4, 2-3.5, 2-3, 2-2.5, 2.5-5, 2.5-4.5, 2.5-4, 2.5-3.5, 2.5-3, 3-5, 3-4.5, 3-4, 3-3.5, 3.5-5, 3.5-4.5, 3.5-4, 4-5, 4-4.5, or 4.5-5.

In some aspects, the dentifrice compositions comprise one or more sweeteners in an amount of 0 wt. % or greater than 0 wt. %. In some aspects, the dentifrice compositions comprise one or more sweeteners in an amount of 5 wt. % or less. In some aspects, the dentifrice compositions comprise one or more surfactants in an amount (wt. %) of 0-5, 0-4.5, 0-4, 0-3.5, 0-3, 0-2.5, 0-2, 0-1.5, 0-1, 0-0.5, 0-0.1, >0-5, >0-4.5, >0-4, >0-3.5, >0-3, >0-2.5, >0-2, >0-1.5, >0-1, >0-0.5, >0-0.1, 0.1-5, 0.1-4.5, 0.1-4, 0.1-3.5, 0.1-3, 0.1-2.5, 0.1-2, 0.1-1.5, 0.1-1, 0.1-0.5, 0.5-5, 0.5-4.5, 0.5-4, 0.5-3.5, 0.5-3, 0.5-2.5, 0.5-2, 0.5-1.5, 0.5-1, 1-5, 1-4.5, 1-4, 1-3.5, 1-3, 1-2.5, 1-2, 1-1.5, 1.5-5, 1.5-4.5, 1.5-4, 1.5-3.5, 1.5-3, 1.5-2.5, 1.5-2, 2-5, 2-4.5, 2-4, 2-3.5, 2-3, 2-2.5, 2.5-5, 2.5-4.5, 2.5-4, 2.5-3.5, 2.5-3, 3-5, 3-4.5, 3-4, 3-3.5, 3.5-5, 3.5-4.5, 3.5-4, 4-5, 4-4.5, or 4.5-5. In some aspects, the dentifrice composition is substantially free of, or does not contain, a sweetener.

In some aspects, the dentifrice compositions comprise one or more whitening agents. In some aspects, the one or more whitening agents comprise titanium dioxide, hydrogen peroxide, sodium tripolyphosphates, or any combination thereof. In some aspects, the dentifrice compositions comprise one or more whitening agents in an amount of 0 wt. % or greater than 0 wt. %. In some aspects, the dentifrice compositions comprise one or more whitening agents in an amount of 2 wt. % or less. In some aspects, the dentifrice compositions comprise one or more whitening agents in an amount (wt. %) of 0-2, 0-1.5, 0-1, 0-0.5, 0-0.1, >0-2, >0-1.5, >0-1, >0-0.5, >0-0.1, 0.1-2, 0.1-1.5, 0.1-1, 0.1-0.5, 0.5-2, 0.5-1.5, 0.5-1, 1-2, 1-1.5, or 1.5-2. In some aspects, the dentifrice compositions do not contain a whitening agent.

In some aspects, the dentifrice compositions comprise one or more abrasives. In some aspects, the one or more abrasives comprise silica, zinc orthophosphate, sodium bicarbonate, alumina, calcium carbonate, calcium pyrophosphate, or any combination thereof. Although the glass compositions disclosed herein may have an abrasive effect in the dentifrice compositions, the glass compositions are not considered an abrasive herein for purposes of the amounts or inclusion of the one or more abrasives. In some aspects, the dentifrice compositions comprise one or more abrasives in an amount of 0 wt. % or greater than 0 wt. %. In some aspects, the dentifrice compositions comprise one or more abrasives in an amount of 20 wt. % or less. In some aspects, the dentifrice compositions comprise one or more abrasives in an amount (wt. %) of 0-20, 0-15, 0-10, 0-5, 0-1, 1-20, 1-15, 1-10, 1-5, 5-20, 5-15, 5-10, 10-20, 10-15, or 15-20. In some aspects, the dentifrice compositions do not contain an abrasive.

In some aspects, the dentifrice compositions may comprise other ingredients that contribute in some way to a desired dentifrice composition. For example, in some aspects, the dentifrice compositions can comprise one or more of a colorant, a fragrance, a medication, a preservative, a desensitizing agent (e.g., potassium salts such as potassium nitrate), a pH modifiers, a preservative, a tartar control agent, and so forth, or any combination thereof. In some aspects, the dentifrice compositions comprise one or more other ingredients in an amount of 0 wt. % or greater than 0 wt. %. In some aspects, the dentifrice compositions comprise one or more other ingredients in an amount of 10 wt. % or less. In some aspects, the dentifrice compositions comprise one or more other ingredients in an amount (wt. %) of 0-10, 0-9, 0-8, 0-7, 0-6, 0-5, 0-4, 0-3, 0-2, 0-1, 0-0.5, >0-10, >0-9, >0-8, >0-7, >0-6, >0-5, >0-4, >0-3, >0-2, >0-1, >0-0.5, 0.5-10, 0.5-9, 0.5-8, 0.5-7, 0.5-6, 0.5-5, 0.5-4, 0.5-3, 0.5-2, 0.5-1, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10. The amounts of the one or more ingredients can be used to describe the amount of any one of the other ingredients, or can be used to describe the sum of any two or more of the other ingredients.

In some aspects, the dentifrice compositions comprise:
>0-20 wt. % of a glass composition, based on total weight of the matrix,
solvent, and
one or more of a humectant, a fluoride ion source, a flavoring, a surfactant, a sweetener, a whitening agent, an abrasive, or any combination thereof.

In some aspects, the dentifrice compositions comprise:
>0-20 wt. % of a glass composition, based on total weight of the matrix,
water, and
one or more of a humectant, a fluoride ion source, a flavoring, a surfactant, a sweetener, a whitening agent, an abrasive, or any combination thereof.

In some aspects, the dentifrice compositions comprise:
>0-20 wt. % of a glass composition, based on total weight of the matrix,
glycerin, and
one or more of a humectant, a fluoride ion source, a flavoring, a surfactant, a sweetener, a whitening agent, an abrasive, or any combination thereof.

In some aspects, disclosed is a method for making the matrix, comprising combining the glass composition with a carrier. In some aspects, the carrier is a toothpaste, mouthwash, gel, varnish, rinse, spray, ointment, salve, cream, bandage, polymer film, oral formulation, pill, capsule, or transdermal formulation.

In some aspects, disclosed is a method comprising applying the matrix to a bone or tooth. In some aspects, disclosed is a method comprising applying the matrix to a bone or tooth. In some aspects, this method is performed by an end user, for example, by way of brushing teeth with a toothpaste or swishing a mouth with a mouthwash. In some aspects, this method is performed by a professional, such as a doctor, a dentist, or other oral care professional, by way of applying the matrix to a bone, brushing a patient's teeth, directing a patient to swish their mouth with a mouthwash, applying the matrix to patient's teeth, and so forth.

In some aspects, the method of applying the matrix to a bone or tooth remineralizes the bone or tooth. In some aspects, the method of applying the matrix to a tooth treats caries of the tooth, treats dentin hypersensitivity of the tooth, or any combination thereof. In some aspects, the method of applying the matrix to a tooth remineralizes the tooth (e.g., by forming fluorapatite in or on the tooth, such as in or on the enamel of the tooth). In some aspects, the method of applying the matrix to a tooth treats caries of the tooth, treats dentin hypersensitivity of the tooth, or any combination thereof, remineralizes the tooth (e.g., enamel) by forming fluorapatite in or on the tooth (e.g., enamel). In some aspects, the method is facilitated by contact with saliva in a mouth (e.g., a patient's mouth).

Various aspects are contemplated herein, several of which are set forth in the paragraphs below. It is explicitly contemplated that any aspect or portion thereof can be combined to form a combination.

Aspect 1: A glass composition, comprising:
15-45 wt. % SiO₂;
10-30 wt. % P₂O₅;
30-60 wt. % CaO;
0.5-15 wt. % ZrO₂;
0.1-10 wt. % F;
0-25 wt. % MgO;
0-5 wt. % B₂O₃;
0-5 wt. % Al₂O₃;
0-10 wt. % Li₂O;
0-10 wt. % Na₂O;
0-10 wt. % K₂O;
0-10 wt. % SrO;
0-10 wt. % ZnO; and
Li₂O+Na₂O+K₂O of less than or equal to 10 wt. %;
based on total weight of the glass composition.

Aspect 2: The glass composition of aspect 1, or any preceding aspect, wherein the glass composition is a glass-ceramic comprising a fluorapatite crystal phase.

Aspect 3: The glass composition of aspect 2, or any preceding aspect, wherein the fluorapatite crystal phase is the major crystalline phase.

Aspect 4: The glass composition of aspect 2 or 3, or any preceding aspect, further comprising at least one minor crystalline phase of calcium silicate, calcium phosphate, calcium fluoride, or any combination thereof.

Aspect 5: The glass composition of any one of aspects 1-4, or any preceding aspect, wherein the glass composition is a glass-ceramic comprising a glass phase in an amount of at least 5 wt. %, based on total weight of the glass-ceramic.

Aspect 6: The glass composition of any one of aspects 1-5, or any preceding aspect, wherein the glass composition is a glass-ceramic comprising a total crystallinity of 5-50 wt. %.

Aspect 7: The glass composition of any one of aspects 1-6, or any preceding aspect, wherein the F is derived from calcium fluoride, magnesium fluoride, sodium fluoride, potassium fluoride, stannous fluoride, sodium monofluorophosphate, sodium difluorophosphate, or any combination thereof.

Aspect 8: The glass composition of any one of aspects 1-7, or any preceding aspect, comprising:
25-45 wt. % SiO₂;
10-25 wt. % P₂O₅;
37-50 wt. % CaO;
0.5-8 wt. % ZrO₂;
0.1-5 wt. % F;
>0-10 wt. % MgO;
based on total weight of the glass composition.

Aspect 9: The glass composition of any one of aspects 1-8, or any preceding aspect, comprising Li₂O+Na₂O+K₂O in an amount of less than or equal to 0.5 wt. %, based on total weight of the glass composition.

Aspect 10: The glass composition of any one of aspects 1-9, or any preceding aspect, comprising:
at least one of:
   >0-25 wt. % MgO;
   >0-5 wt. % B₂O₃;
   >0-5 wt. % Al₂O₃;
   >0-10 wt. % Li₂O;
   >0-10 wt. % Na₂O;
   >0-10 wt. % K₂O;
   >0-10 wt. % SrO; or
   >0-10 wt. % ZnO;
provided that Li₂O+Na₂O+K₂O is less than or equal to 10 wt. %;
based on total weight of the glass composition.

Aspect 11: The glass composition of any one of aspects 1-10, or any preceding aspect, wherein the glass composition is in a form of particulates, microbeads, fibers, or a combination thereof.

Aspect 12: A matrix comprising the glass composition of any one of aspects 1-11, or any preceding aspect, wherein:
the glass composition is attached to the matrix or mixed therein, and
the matrix includes at least one of: a toothpaste, mouthwash, gel, varnish, rinse, spray, ointment, salve, cream, bandage, polymer film, oral formulation, pill, capsule, or transdermal formulation.

Aspect 13: The matrix of aspect 12, or any preceding aspect, wherein the matrix is a dentifrice composition.

Aspect 14: The matrix of aspect 13, or any preceding aspect, wherein the dentifrice composition comprises:
>0-20 wt. % of the glass composition, based on total weight of the matrix,
solvent, and
one or more of a humectant, a fluoride ion source, a flavoring, a surfactant, a sweetener, a whitening agent, an abrasive, or any combination thereof.

Aspect 15: A method for making the matrix of any one of aspects 12-14, or any preceding aspect, comprising combining a carrier with the glass composition.

Aspect 16: A method comprising applying to a tooth the dentifrice composition of aspect 13 or 14, or any preceding aspect.

Aspect 17: The method of aspect 16, or any preceding aspect, wherein the method remineralizes enamel of a tooth, treats caries of a tooth, treats dentin hypersensitivity of the tooth, or any combination thereof.

Aspect 18: A method for making the glass composition of any one of aspects 1-11, or any preceding aspect, comprising:
combining each component of the glass composition to form a mixture;
heating the mixture to a temperature of 1500° C. or less to form a melted mixture; and
cooling the melted mixture to form a cooled mixture.

Aspect 19: The method of aspect 18, or any preceding aspect, wherein the cooling step spontaneously results in at least partial crystallization into a fluorapatite crystal phase.

Aspect 20: The method of aspect 18 or 19, or any preceding aspect, wherein, after the cooling step, the cooled mixture is not heated to a crystallization temperature.

Aspect 21: The method of any one of aspects 18-20, or any preceding aspect, further comprising forming the cooled mixture into particulates, microbeads, or a combination thereof.

Aspect 22: A combination of any two or more of aspects 1-21, or any one or more portions thereof.

### Example

The following examples illustrate non-limiting aspects of the disclosure and are not intended to be limiting on the scope of the disclosure or claims.

Non-limiting examples of amounts of precursor oxides for forming the glass compositions are listed in Table 2.

TABLE 2

| Oxide | | | | | | | | | | |
|---|---|---|---|---|---|---|---|---|---|---|
| (wt. %) | 1 | 2 | 3 | 4 | 5 | 6 | 7 | 8 | 9 | 10 |
| SiO₂ | 33.5 | 33.5 | 33.5 | 33.5 | 41 | 31.5 | 41.5 | 28.5 | 30 | 38.5 |
| MgO | 4.5 | 4.5 | 4.5 | 4.5 | 4.5 | 1 | 4.5 | 4.5 | 4.5 | 4.5 |
| CaO | 42.5 | 42.5 | 42.5 | 42.5 | 30 | 42.5 | 42.5 | 42.5 | 42.5 | 42.5 |
| P₂O₅ | 15 | 14.5 | 13.5 | 11.5 | 20 | 15 | 10 | 20 | 15 | 10 |
| ZrO₂ | 4 | 4 | 4 | 4 | 4 | 6 | 1 | 4 | 4 | 4 |
| F⁻ | 0.5 | 1 | 2 | 4 | 0.5 | 4 | 0.5 | 0.5 | 4 | 0.5 |

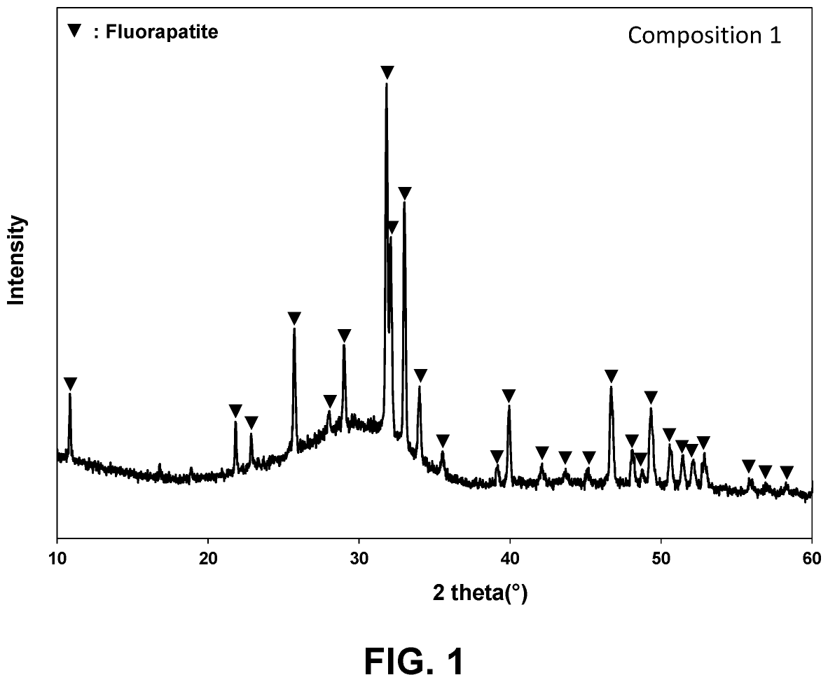

The glass compositions of Table 2 were prepared by combining each component of the glass composition to form a mixture, heating the mixture to a temperature of 1500° C. or less, and then cooling the melted mixture, which spontaneously resulted in at least partial crystallization into a fluorapatite crystal phase (see, e.g., FIG. 1). In this regard, typical glass compositions generally do not spontaneously crystallize upon cooling, but instead generally require an additional heat treatment step of the cooled mixture in order to form a crystalline phase. However, with the glass compositions disclosed herein, the additional heat treatment step of the cooled mixture is not necessary, since upon cooling of the original melted mixture, a crystalline phase of fluorapatite forms spontaneously.

In this regard, FIG. 1 illustrates powder x-ray diffraction (XRD) analysis of composition 1 in Table 2 prepared in accordance with the disclosures herein. The location of XRD peaks corresponding to fluorapatite formation are shown in FIG. 1. Powder XRD analysis was conducted by preparing the samples by first drying and then grinding to a fine powder using a Rocklabs ring mill. The powder was then analyzed using a Bruker D4 Endeavor device equipped with a LynxEye™ silicon strip detector. X-ray scanning was conducted from 5° to 80° (2θ) for data collection.

As used herein, the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination.

References herein to the positions of elements (e.g., “top,” “bottom,” “above,” “below,” “first,” “second,” etc.) are merely used to describe the orientation of various elements in the FIGURES. It should be noted that the orientation of various elements may differ according to other exemplary embodiments, and that such variations are intended to be encompassed by the present disclosure. Moreover, these relational terms are used solely to distinguish one entity or action from another entity or action, without necessarily requiring or implying any actual such relationship or order between such entities or actions.

Modifications of the disclosure will occur to those skilled in the art and to those who make or use the disclosure. Therefore, it is understood that the embodiments shown in the drawings and described above are merely for illustrative purposes and not intended to limit the scope of the disclosure, which is defined by the following claims, as interpreted according to the principles of patent law, including the doctrine of equivalents.

It will be understood by one having ordinary skill in the art that construction of the described disclosure, and other components, is not limited to any specific material. Other exemplary embodiments of the disclosure disclosed herein may be formed from a wide variety of materials, unless described otherwise herein.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the invention as recited in the appended claims.

As utilized herein, “optional,” “optionally,” or the like are intended to mean that the subsequently described event or circumstance can or cannot occur, and that the description includes instances where the event or circumstance occurs and instances where it does not occur. As used herein, the indefinite articles “a,” “an,” and the corresponding definite article “the” mean “at least one” or “one or more,” unless otherwise specified. It also is understood that the various features disclosed in the specification and the drawings can be used in any and all combinations.

With respect to the use of substantially any plural and/or singular terms herein, those having skill in the art can translate from the plural to the singular and/or from the singular to the plural as is appropriate to the context and/or application. The various singular/plural permutations may be expressly set forth herein for the sake of clarity.

Unless otherwise specified, all compositions are expressed in terms of as-batched weight percent (wt. %). As will be understood by those having ordinary skill in the art, various melt constituents (e.g., silicon, alkali- or alkaline-based, boron, etc.) may be subject to different levels of volatilization (e.g., as a function of vapor pressure, melt time and/or melt temperature) during melting of the constituents. As such, the as-batched weight percent values used in relation to such constituents are intended to encompass values within ±0.5 wt. % of these constituents in final, as-melted articles. With the forgoing in mind, substantial compositional equivalence between final articles and as-batched compositions is expected.

It will be apparent to those skilled in the art that various modifications and variations can be made without departing from the spirit or scope of the claimed subject matter. Accordingly, the claimed subject matter is not to be restricted except in light of the attached claims and their equivalents.

What is claimed is:

1. A glass composition, comprising:
15-45 wt. % SiO₂;
10-30 wt. % P₂O₅;
30-60 wt. % CaO;
0.5-15 wt. % ZrO₂;
0.1-10 wt. % F⁻;
0-25 wt. % MgO;
0-5 wt. % B₂O₃;
0-5 wt. % Al₂O₃;
0-10 wt. % Li₂O;
0-10 wt. % Na₂O;
0-10 wt. % K₂O;
0-10 wt. % SrO;
0-10 wt. % ZnO; and
Li₂O+Na₂O+K₂O of less than or equal to 10 wt. %;
based on total weight of the glass composition.

2. The glass composition of claim 1, wherein the glass composition is a glass-ceramic comprising a fluorapatite crystal phase.

3. The glass composition of claim 2, wherein the fluorapatite crystal phase is the major crystalline phase.

4. The glass composition of claim 2, further comprising at least one minor crystalline phase of calcium silicate, calcium phosphate, calcium fluoride, or any combination thereof.

5. The glass composition of claim 1, wherein the glass composition is a glass-ceramic comprising a glass phase in an amount of at least 5 wt. %, based on total weight of the glass-ceramic.

6. The glass composition of claim 1, wherein the glass composition is a glass-ceramic comprising a total crystallinity of 5-50 wt. %.

7. The glass composition of claim 1, wherein the F is derived from calcium fluoride, magnesium fluoride, sodium fluoride, potassium fluoride, stannous fluoride, sodium monofluorophosphate, sodium difluorophosphate, or any combination thereof.

8. The glass composition of claim 1, comprising:
25-45 wt. % SiO₂;
10-25 wt. % P₂O₅;
37-50 wt. % CaO;
0.5-8 wt. % ZrO₂;
0.1-5 wt. % F;
>0-10 wt. % MgO;
based on total weight of the glass composition.

9. The glass composition of claim 1, comprising Li₂O+Na₂O+K₂O in an amount of less than or equal to 0.5 wt. %, based on total weight of the glass composition.

10. The glass composition of claim 1, comprising:
at least one of:
>0-25 wt. % MgO;
>0-5 wt. % B₂O₃;
>0-5 wt. % Al₂O₃;
>0-10 wt. % Li₂O;
>0-10 wt. % Na₂O;
>0-10 wt. % K₂O;
>0-10 wt. % SrO; or
>0-10 wt. % ZnO;
provided that Li₂O+Na₂O+K₂O is less than or equal to 10 wt. %;
based on total weight of the glass composition.

11. The glass composition of claim 1, wherein the glass composition is in a form of particulates, microbeads, fibers, or a combination thereof.

12. A matrix comprising the glass composition of claim 1, wherein:
the glass composition is attached to the matrix or mixed therein, and
the matrix includes at least one of: a toothpaste, mouthwash, gel, varnish, rinse, spray, ointment, salve, cream, bandage, polymer film, oral formulation, pill, capsule, or transdermal formulation.

13. The matrix of claim 12, wherein the matrix is a dentifrice composition.

14. The matrix of claim 13, wherein the dentifrice composition comprises:
>0-20 wt. % of the glass composition, based on total weight of the matrix,
solvent, and
one or more of a humectant, a fluoride ion source, a flavoring, a surfactant, a sweetener, a whitening agent, an abrasive, or any combination thereof.

15. A method for making the matrix of claim 12, comprising combining a carrier with the glass composition.

16. A method comprising applying to a tooth the dentifrice composition of claim 13.

17. The method of claim 16, wherein the method remineralizes enamel of a tooth, treats caries of a tooth, treats dentin hypersensitivity of the tooth, or any combination thereof.

18. A method for making the glass composition of claim 1, comprising:
combining each component of the glass composition to form a mixture;
heating the mixture to a temperature of 1500° C. or less to form a melted mixture; and
cooling the melted mixture to form a cooled mixture.

19. The method of claim 18, wherein the cooling step spontaneously results in at least partial crystallization into a fluorapatite crystal phase.

20. The method of claim 18, wherein, after the cooling step, the cooled mixture is not heated to a crystallization temperature.
